(19) United States
(12) Patent Application Publication
(10) Pub. No.: US 2024/0277239 A1
(43) Pub. Date: Aug. 22, 2024

(54) SPHYGMOMANOMETER, CONTROL DEVICE, AND NON-TRANSITORY COMPUTER-READABLE MEDIUM

(71) Applicant: NIHON KOHDEN CORPORATION, Tokyo (JP)

(72) Inventor: Shogo TOTSUKA, Tokorozawa-shi, Saitama (JP)

(73) Assignee: NIHON KOHDEN CORPORATION, Tokyo (JP)

(21) Appl. No.: 18/436,849

(22) Filed: Feb. 8, 2024

(30) Foreign Application Priority Data
Feb. 17, 2023 (JP) 2023-023399

(51) Int. Cl. A61B 5/0225; A61B 5/022

(52) U.S. Cl. CPC A61B 5/0225; A61B 5/02233

(57) ABSTRACT

A control device is configured to: cause an inflating/deflating device to initiate a first air feeding to a cuff with a prescribed flow rate; calculate an estimated value of a capacity volume of the cuff based on a time length for which the internal pressure inflates from a first value to a second value; cause the inflating/deflating device to perform at least air exhausting before an inflation of the internal pressure reaches a steady state to deflate the internal pressure; determine, based on the estimated value, an air feeding flow rate for obtaining a prescribed inflating speed; cause the inflating/deflating device to initiate a second air feeding to the cuff with the air feeding flow rate before the internal pressure reaches zero; and measure blood pressure of the subject while the second air feeding is performed.

### CROSS-REFERENCE TO RELATED APPLICATION

The present application is based on Japanese Patent Application No. 2023-023399 filed on Feb. 17, 2023, the entire contents of which are incorporated herein by reference.

### BACKGROUND

The presently disclosed subject matter relates to a sphygmomanometer. The presently disclosed subject matter also relates to a control device installed in the sphygmomanometer, as well as a non-transitory computer-readable medium having stored a computer program adapted to be executable by a processor installed in the sphygmomanometer.

Japanese Patent Publication No. 2013-090825A discloses an inflation-type sphygmomanometer that non-invasively measures blood pressure of a subject while inflating internal pressure of a cuff that is attached to a body of the subject.

### SUMMARY

It is demanded to enhance the measurement accuracy of the inflation-type sphygmomanometer.

An illustrative aspect of the presently disclosed subject matter provides a sphygmomanometer, comprising:
a ventilation passage adapted to be communicated with an interior of a cuff that is adapted to be attached to a body of a subject;
an inflating/deflating device configured to perform at least one of air feeding and air exhausting through the ventilation passage to inflate/deflate internal pressure of the cuff; and
a control device configured to control an operation of the inflating/deflating device,
wherein the control device is configured to:
   cause the inflating/deflating device to initiate a first air feeding to the cuff with a prescribed flow rate;
   calculate an estimated value of a capacity volume of the cuff based on a time length for which the internal pressure inflates from a first value to a second value;
   cause the inflating/deflating device to perform at least the air exhausting before an inflation of the internal pressure reaches a steady state to deflate the internal pressure;
   determine, based on the estimated value, an air feeding flow rate for obtaining a prescribed inflating speed;
   cause the inflating/deflating device to initiate a second air feeding to the cuff with the air feeding flow rate before the internal pressure reaches zero; and
   measure blood pressure of the subject while the second air feeding is performed.

An illustrative aspect of the presently disclosed subject matter provides a control device adapted to be installed in a sphygmomanometer, comprising:
an interface configured to receive a signal corresponding to an inner pressure of a cuff that is adapted to be attached to a body of a subject; and
a processor configured to control an operation of an inflating/deflating device that is configured to inflate/deflate the inner pressure of the cuff by performing at least one of air feeding and air exhausting through a ventilation passage that is adapted to be communicated with an interior of the cuff,
wherein the processor is configured to:
   cause the inflating/deflating device to initiate a first air feeding to the cuff with a prescribed flow rate;
   calculate an estimated value of a capacity volume of the cuff based on a time length for which the internal pressure inflates from a first value to a second value;
   cause the inflating/deflating device to perform at least the air exhausting before an inflation of the internal pressure reaches a steady state to deflate the internal pressure;
   determine, based on the estimated value, an air feeding flow rate for obtaining a prescribed inflating speed;
   cause the inflating/deflating device to initiate a second air feeding to the cuff with the air feeding flow rate before the internal pressure reaches zero; and
   measure blood pressure of the subject while the second air feeding is performed.

An illustrative aspect of the presently disclosed subject matter provides a non-transitory computer-readable medium having stored a computer program adapted to be executed by a processor installed in a sphygmomanometer, the computer program being configured, when executed, to cause the sphygmomanometer to:
initiate a first air feeding with a prescribed flow rate to a cuff that is adapted to be attached to a body of a subject;
calculate an estimated value of a capacity volume of the cuff based on a time length for which the internal pressure inflates from a first value to a second value;
perform at least the air exhausting before an inflation of the internal pressure reaches a steady state to deflate the internal pressure;
determine, based on the estimated value, an air feeding flow rate for obtaining a prescribed inflating speed;
initiate a second air feeding to the cuff with the air feeding flow rate before the internal pressure reaches zero; and
measure blood pressure of the subject while the second air feeding is performed.

In order to measure the blood pressure of the subject non-invasively, it is necessary to detect fluctuations of the internal pressure of the cuff caused by the pulse wave of the subject. In order to accurately detect the internal pressure fluctuations, it is necessary to keep the inflating speed of the cuff constant. In order to determine an adequate inflating speed, the capacity volume of the cuff is estimated through the first air feeding. However, since the inflating speed is not constant during the execution of the first air feeding, a meaningful blood pressure measurement cannot be performed. In other words, a meaningful blood pressure measurement cannot be performed in a pressure range that is less than the second value required for estimating the capacity volume of the cuff. Accordingly, the inflation-type blood pressure measurement cannot be performed on a subject whose blood pressure is in such a pressure range.

On the other hand, according to the configuration of each of the above illustrative aspects, since the cuff is subjected to the deflation process after the capacity volume is estimated, it is possible to perform the inflation-type blood pressure measurement even for a subject with a low blood pressure that falls the range between the second value and the third value. In addition, the inventor of the present application has found that, when the internal pressure of the cuff is deflated to zero, the inflating speed of the cuff by the second air feeding that is subsequently performed is hardly stabilized. In each of the above illustrative aspects, since the second air feeding is initiated before the internal pressure of the cuff that is subjected to the deflation reaches zero, it is possible to suppress fluctuations of the inflating speed of the cuff. As a result, the measurement accuracy of the inflation-type sphygmomanometer can be enhanced.

### DESCRIPTION OF THE DRAWINGS

FIG. 1 illustrates a functional configuration of a sphygmomanometer according to one exemplary embodiment.

FIG. 2 illustrates an exemplary flow of processing executed by a processor of FIG. 1.

FIG. 3 illustrates changes with time of an internal pressure of a cuff in accordance with the processing of FIG. 2.

### DESCRIPTION OF EMBODIMENTS

Exemplary embodiments will be described in detail with reference to the accompanying drawings.

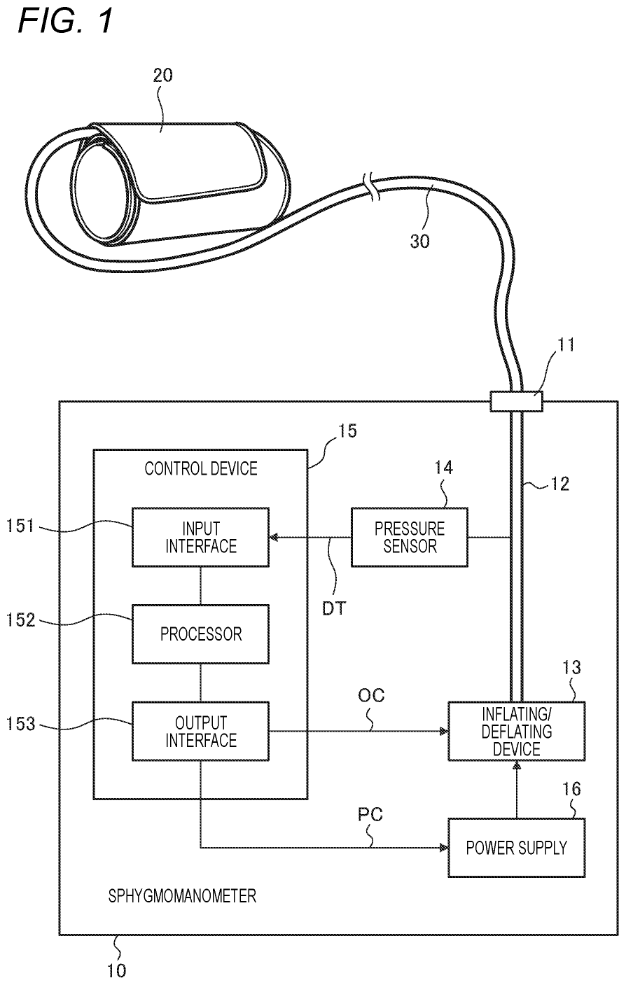

FIG. 1 illustrates a functional configuration of a sphygmomanometer 10 according to an exemplary embodiment. The sphygmomanometer 10 is a device for non-invasively measuring blood pressure of a subject with a cuff 20. A prescribed portion of a body of the subject is wrapped with the cuff 20 when used.

The sphygmomanometer 10 includes a connector 11. A tube 30 communicating with the interior of the cuff 20 is connected to the connector 11.

The sphygmomanometer 10 includes a ventilation passage 12. The ventilation passage 12 is configured to allow a fluid such as air to flow through the interior of the ventilation passage 12. The ventilation passage 12 communicates with the connector 11. One end of the tube 30 is connected to the connector 11, whereby the ventilation passage 12 is communicated with the interior of the cuff 20.

The sphygmomanometer 10 includes an inflating/deflating device 13. The inflating/deflating device 13 is configured to inflate or deflate the internal pressure of the cuff 20 by executing at least one of air feeding to the cuff 20 through the ventilation passage 12 and air exhausting through the ventilation passage 12.

Specifically, the inflating/deflating device 13 includes a pump mechanism and a valve mechanism. The pump mechanism is configured to inflate the internal pressure of the cuff 20 by feeding air to the cuff 20 through the ventilation passage 12. The valve mechanism is configured to establish or cancel communication between the ventilation passage 12 and ambient air.

When the pump mechanism is deactivated and the ventilation passage 12 is communicated with the ambient air by the valve mechanism, the internal pressure of the cuff 20 is deflated. When the pump mechanism is activated and the ventilation passage 12 is communicated with the ambient air by the valve mechanism, the inflating speed of the internal pressure of the cuff 20 is decreased.

The sphygmomanometer 10 includes a pressure sensor 14. The pressure sensor 14 is configured to detect pressure in the ventilation passage 12. The pressure substantially matches the internal pressure of the cuff 20. Accordingly, it can be said that the pressure sensor 14 detects the internal pressure of the cuff 20. The pressure sensor 14 is configured to output a detection signal DT corresponding to the detected pressure. The detection signal DT may be an analog signal or a digital signal in accordance with the specification of the pressure sensor 14.

The sphygmomanometer 10 includes a control device 15. The control device 15 is configured to control the operation of the inflating/deflating device 13. Specifically, the control device 15 includes an input interface 151, a processor 152, and an output interface 153.

The input interface 151 is configured as a hardware interface that receives the detection signal DT from the pressure sensor 14. In a case where the detection signal DT is an analog signal, the input interface 151 is provided with an adequate conversion circuit including an A/D converter.

The processor 152 is configured to output, from the output interface 153, an operation control signal OC that causes the inflating/deflating device 13 to perform an intended operation. The operation control signal OC may be an analog signal or a digital signal in accordance with the specification of the inflating/deflating device 13. The output interface 153 is configured as a hardware interface. In a case where the operation control signal OC is an analog signal, the output interface 153 is provided with an adequate conversion circuit including a D/A converter. This description is similarly applied to other signals that can be outputted by the output interface 153 described later.

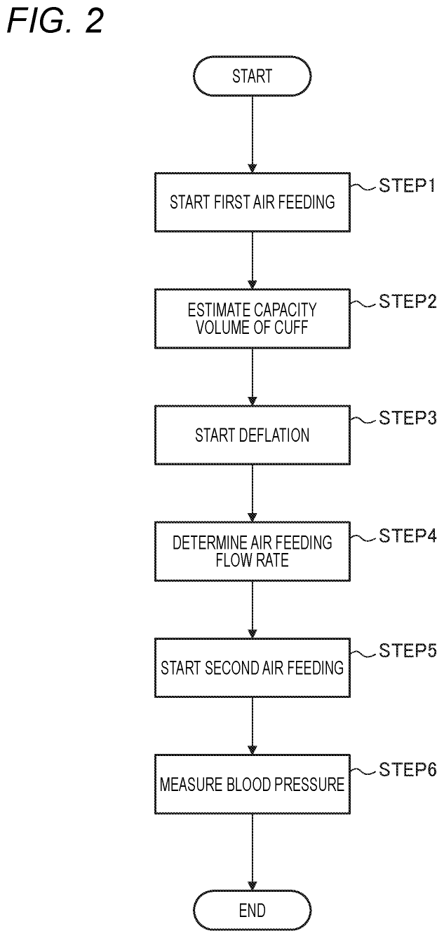

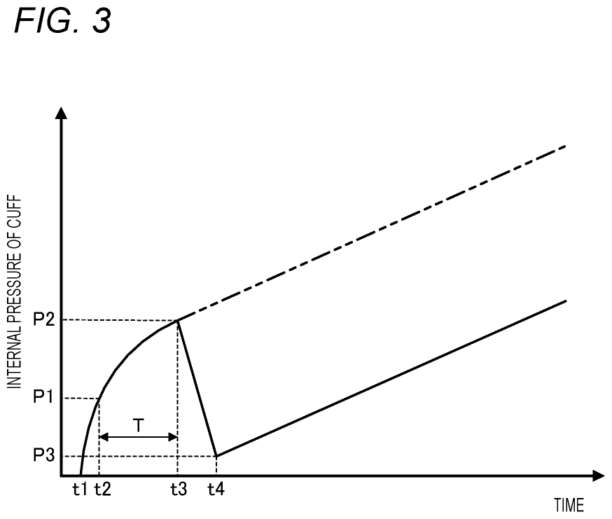

With reference to FIGS. 2 and 3, exemplary processing executed by the processor 152 will be described in detail.

The processor 152 first executes a first air feeding process (STEP 1 in FIG. 2). Specifically, the processor 152 outputs, from the output interface 153, an operation control signal OC that causes the inflating/deflating device 13 to start feeding air to the cuff 20 at a prescribed flow rate. The flow rate of the air that is fed to the cuff 20 decreases in accordance with an inflation of the internal pressure of the cuff 20.

In FIG. 3, the first air feeding by the inflating/deflating device 13 is initiated at a time point t1. As a result, the internal pressure of the cuff 20 is inflated. In a case where air is fed at a constant flow rate, the inflating speed of the internal pressure starts to decrease gradually. Subsequently, the processor 152 executes a process of calculating an estimated value of a capacity volume of the cuff 20 while causing the inflating/deflating device 13 to perform the first air feeding (STEP 2 in FIG. 2). Specifically, an estimated value of the capacity volume of the cuff 20 is calculated based on a time length T in FIG. 3. The time length T corresponds to a time period from a time point t2 when the internal pressure of the cuff 20 has a first value P1 to a time point t3 when the internal pressure of the cuff 20 has a second value P2. The first value P1 and the second value P2 may be specified based on the detection signal DT from the pressure sensor 14.

The first value P1 and the second value P2 are prescribed. The time points t2 and t3 change in accordance with the capacity volume of the cuff 20. Accordingly, the time length T also varies in accordance with the capacity volume of the cuff 20. In general, the larger the capacity volume of the cuff 20, the longer the time length T. Namely, the capacity volume V of the cuff 20 is expressed by the following expression as a function of the time length T.

$$V = f 1 ( T )$$

The function f1 is determined in advance by acquiring a number of samples in which each of the capacity volume of the cuff, the subject to whom the cuff is to be attached, and the way of wrapping the cuff around the body of the subject is made different one another, and applying statistical processing. The function f1 is stored in a memory (not illustrated) so that the processor 152 can refer to the function f1. The processor 152 acquires the time length T based on the detection signal DT outputted from the pressure sensor 14, and calculates the estimated value of the capacity volume V of the cuff 20 by substituting the acquired time length T into the function f1.

Subsequently, the processor 152 executes a process of deflating the cuff 20 (STEP 3 in FIG. 2). Specifically, the processor 152 outputs, from the output interface 153, an operation control signal OC that causes the inflating/deflating device 13 to perform at least air exhausting.

The deflation is performed before the internal pressure of the cuff 20 reaches a steady state. As used herein, the term “steady state of the internal pressure” means a state where no substantial change with time in the value of the internal pressure is observed. In FIG. 3, the deflation is initiated at a time point t3 when the internal pressure of the cuff 20 has the second value P2. However, as long as the deflation is initiated before the internal pressure reaches the steady state, the time point when the deflation is initiated may be different from the time point when the internal pressure has the second value P2.

Subsequently, the processor 152 executes a process of determining an air feeding flow rate for realizing a prescribed inflating speed of the cuff 20 based on the calculated estimated value of the capacity volume of the cuff 20 (STEP 4 in FIG. 2).

In order to measure the blood pressure of the subject non-invasively, it is necessary to detect fluctuations of the internal pressure of the cuff 20 caused by the pulse wave of the subject. In order to accurately detect the internal pressure fluctuations, it is necessary to keep the inflating speed of the cuff 20 constant. The inflating speed depends on the air feeding flow rate and the capacity volume of the cuff. For example, in a case where the air feeding flow rate is constant, the inflating speed is decreased as the capacity volume of the cuff is increased.

The sphygmomanometer 10 according to the present exemplary embodiment is configured such that the inflation is performed at a constant speed regardless of the capacity volume of the cuff 20. The “constant inflating speed” is determined in advance as a value at which a blood pressure measurement can be appropriately performed as intended. As used herein, the expression “constant inflating speed” does not mean that no change in the inflating speed is strictly required, but rather means that it allows fluctuations of the inflating speed to an extent that a meaningful blood pressure measurement can be performed. Examples of the acceptable fluctuation range include less than +3 mmHg per second.

Accordingly, an air feeding flow rate F for obtaining a prescribed inflating speed is expressed by the following expression as a function of the capacity volume V of the cuff 20.

$$F = f 2 ( V )$$

The function f2 is determined in advance based on the above-described policy. The function f2 is stored in a memory (not illustrated) so that the processor 152 can refer to the function f2. The processor 152 determines the air feeding flow rate F by substituting the estimated value of the capacity volume of the cuff 20 that is calculated in STEP 2, in the function f2.

The process of determining the air feeding flow rate F (STEP 4) may be performed prior to the process of deflating the cuff 20 (STEP 3), or may be performed in parallel with the process of deflating the cuff 20 (STEP 3).

Subsequently, the processor 152 executes a second air feeding process (STEP 5). Specifically, the processor 152 outputs, from the output interface 153, an operation control signal OC that causes the inflating/deflating device 13 to feed air to the cuff 20 at the flow rate F as determined.

In FIG. 3, the second air feeding by the inflating/deflating device 13 is initiated at a time point t4. The second air feeding is performed before the internal pressure of the cuff 20 subjected to the deflation reaches zero. In this example, the second air feeding is initiated when the internal pressure is deflated to a third value P3. The third value P3 is determined in advance as a value larger than zero. The third value P3 may be specified based on the detection signal DT from the pressure sensor 14. As a result, the internal pressure of the cuff 20 is inflated at a constant speed regardless of the capacity volume thereof.

Subsequently, the processor 152 executes a process of measuring the blood pressure of the subject during the execution of the second air feeding by the inflating/deflating device 13 (STEP 6 in FIG. 2). As described above, the processor 152 measures the blood pressure through the pulse wave fluctuations of the internal pressure of the cuff 20 that is detected by the pressure sensor 14. When the measurement of the blood pressure is completed, the processor 152 outputs, from the output interface 153, an operation control signal OC that causes the inflating/deflating device 13 to perform air exhausting thereby deflating the cuff 20, and then terminates the processing.

The dashed chain line illustrated in FIG. 3 illustrates a case where, after the capacity volume of the cuff 20 is estimated, the inflation is performed at a constant speed without passing through the deflation process. As described above, during a time period in which the inflating speed of the internal pressure of the cuff 20 is not constant (between the time points t1 and t3), a meaningful blood pressure measurement cannot be performed. In other words, a meaningful blood pressure measurement cannot be performed in a pressure range that is less than the second value P2 required for estimating the capacity volume of the cuff 20. Accordingly, the inflation-type blood pressure measurement cannot be performed on a subject whose blood pressure is in such a pressure range.

On the other hand, according to the sphygmomanometer 10 of the present exemplary embodiment, since the cuff 20 is subjected to the deflation process after the capacity volume is estimated, it is possible to perform the inflation-type blood pressure measurement even for a subject with a low blood pressure that falls the range between the second value P2 and the third value P3. In addition, the inventor of the present application has found that, when the internal pressure of the cuff 20 is deflated to zero, the inflating speed of the cuff 20 by the second air feeding that is subsequently performed is hardly stabilized. In the sphygmomanometer 10 according to the present exemplary embodiment, since the second air feeding is initiated before the internal pressure of the cuff 20 that is subjected to the deflation reaches zero, it is possible to suppress fluctuations of the inflating speed of the cuff 20. As a result, the measurement accuracy of the inflation-type sphygmomanometer 10 can be enhanced.

In particular, in a case where the third value P3 is set to less than 10 mmHg, it is possible to perform the inflation-type blood pressure measurement even for a patient with hypotension such as a neonate with cardiac disease.

As illustrated in FIG. 1, the sphygmomanometer 10 includes at least a power supply 16 that supplies driving power to the inflating/deflating device 13. The processor 152 of the control device 15 may be configured to output, from the output interface 153, a power control signal PC that causes the power supply 16 to increase or decrease the power that is supplied to the inflating/deflating device 13.

In the present exemplary embodiment, the deflation of the cuff 20 is performed without interrupting the power supplied from the power supply 16 to the inflating/deflating device 13. For example, the processor 152 outputs, from the output interface 153, a power control signal PC that causes the power supply 16 to supply power to an extent that enables the inflating/deflating device 13 to continue gentle air feeding even when the air exhausting is performed. Alternatively, the processor 152 outputs, from the output interface 153, a power control signal PC that causes the power supply 16 to maintain a value of the power to be supplied so as to be lower than a value that enables the inflating/deflating device 13 to feed air.

According to the above configuration, as compared with a case where the power supply to the inflating/deflating device 13 is resumed after the interruption of the power supply, the transition from the deflation process to the second air feeding process is smoothly performed, so that the fluctuations of the inflating speed in the second air feeding process is easily suppressed. Accordingly, it is possible to enhance the measurement accuracy of the inflation-type sphygmomanometer 10.

The processor 152 of the control device 15 may be configured to output, from the output interface 153, an operation control signal OC that causes the inflating/deflating device 13 to increase the air feeding flow rate after the second air feeding is initiated. The increased amount of the air feeding flow rate is adjusted such that the inflating speed of the cuff 20 is maintained. The increase of the air feeding flow rate may be stepwise or continuous.

In accordance with the capacity volume of the cuff 20, the inflating speed may be decreased during the execution of the second air feeding. According to the above configuration, even in such a case, since the control is performed such that the inflating speed is maintained, it is possible to easily suppress fluctuations of the inflating speed in the second air feeding process. Accordingly, it is possible to enhance the measurement accuracy of the inflation-type sphygmomanometer 10.

The processor 152 of the control device 15 having various functions described above may be implemented by at least one non-exclusive microprocessor that cooperates with a non-exclusive memory. Examples of the non-exclusive microprocessor include a CPU, an MPU, and a GPU. Examples of the non-exclusive memory include a ROM and a RAM. In this case, a computer program that implements the function may be stored in the ROM. The ROM is an example of a non-transitory computer-readable medium having stored a computer program. The non-exclusive microprocessor designates at least a part of the program stored in the ROM, loads the designated program in the RAM, and executes the above-described processing in cooperation with the RAM. The computer program may be pre-installed in a non-exclusive memory, or may be downloaded from an external server device through a communication network, and then installed in the non-exclusive memory. In this case, the external server device is an example of the non-transitory computer-readable medium having stored the computer program.

The processor 152 may be implemented by at least one exclusive integrated circuitry capable of executing the above-described computer program, such as a microcontroller, an ASIC, and an FPGA. In this case, the above-described computer program is pre-installed in a memory element included in the exclusive integrated circuitry. The memory element is an example of the non-transitory computer-readable medium having stored the computer program. The processor 152 may also be implemented by a combination of the non-exclusive microprocessor and the exclusive integrated circuitry.

The various configurations described above are merely illustrative for facilitating understanding of the presently disclosed subject matter. Each of the illustrative configurations may be appropriately modified or combined with another illustrative configuration within the gist of the present disclosure.

1. A sphygmomanometer, comprising:
a ventilation passage adapted to be communicated with an interior of a cuff that is adapted to be attached to a body of a subject;
an inflating/deflating device configured to perform at least one of air feeding and air exhausting through the ventilation passage to inflate/deflate internal pressure of the cuff; and
a control device configured to control an operation of the inflating/deflating device,
wherein the control device is configured to:
cause the inflating/deflating device to initiate a first air feeding to the cuff with a prescribed flow rate;
calculate an estimated value of a capacity volume of the cuff based on a time length for which the internal pressure inflates from a first value to a second value;
cause the inflating/deflating device to perform at least the air exhausting before an inflation of the internal pressure reaches a steady state to deflate the internal pressure;
determine, based on the estimated value, an air feeding flow rate for obtaining a prescribed inflating speed;
cause the inflating/deflating device to initiate a second air feeding to the cuff with the air feeding flow rate before the internal pressure reaches zero; and
measure blood pressure of the subject while the second air feeding is performed.

2. The sphygmomanometer according to claim 1,
wherein the control device is configured to deflate the internal pressure to a value less than 10 mmHg before the second air feeding is initiated.

3. The sphygmomanometer according to claim 1,
wherein the control device is configured to deflate the internal pressure without interrupting power supply to the inflating/deflating device.

4. The sphygmomanometer according to claim 1,
wherein the control device is configured to increase the air feeding flow rate after the second air feeding is initiated to maintain the inflating speed.

5. A control device adapted to be installed in a sphygmomanometer, comprising:
an interface configured to receive a signal corresponding to an inner pressure of a cuff that is adapted to be attached to a body of a subject; and
a processor configured to control an operation of an inflating/deflating device that is configured to inflate/deflate the inner pressure of the cuff by performing at least one of air feeding and air exhausting through a ventilation passage that is adapted to be communicated with an interior of the cuff,
wherein the processor is configured to:
cause the inflating/deflating device to initiate a first air feeding to the cuff with a prescribed flow rate;
calculate an estimated value of a capacity volume of the cuff based on a time length for which the internal pressure inflates from a first value to a second value;
cause the inflating/deflating device to perform at least the air exhausting before an inflation of the internal pressure reaches a steady state to deflate the internal pressure;
determine, based on the estimated value, an air feeding flow rate for obtaining a prescribed inflating speed;
cause the inflating/deflating device to initiate a second air feeding to the cuff with the air feeding flow rate before the internal pressure reaches zero; and
measure blood pressure of the subject while the second air feeding is performed.

6. A non-transitory computer-readable medium having stored a computer program adapted to be executed by a processor installed in a sphygmomanometer, the computer program being configured, when executed, to cause the sphygmomanometer to:
initiate a first air feeding with a prescribed flow rate to a cuff that is adapted to be attached to a body of a subject;
calculate an estimated value of a capacity volume of the cuff based on a time length for which the internal pressure inflates from a first value to a second value;
perform at least the air exhausting before an inflation of the internal pressure reaches a steady state to deflate the internal pressure;
determine, based on the estimated value, an air feeding flow rate for obtaining a prescribed inflating speed;
initiate a second air feeding to the cuff with the air feeding flow rate before the internal pressure reaches zero; and
measure blood pressure of the subject while the second air feeding is performed.
